(19) United States
(12) Patent Application Publication
(10) Pub. No.: US 2019/0170773 A1
(43) Pub. Date: Jun. 6, 2019

(54) Testosterone Saliva Test

(71) Applicant: Dawn Maslar, Davie, FL (US)

(72) Inventor: Dawn Maslar, Davie, FL (US)

(21) Appl. No.: 16/151,284

(22) Filed: Oct. 3, 2018

Related U.S. Application Data
(63) Continuation-in-part of application No. 15/833,702, filed on Dec. 6, 2017.

(51) Int. Cl. G01N 33/74; G01N 21/78; G01N 33/558

(52) U.S. Cl. CPC G01N 33/743; G01N 2021/757; G01N 33/558; G01N 21/78

(57) ABSTRACT

A lateral flow assay format test that accepts a saliva sample that is reacted with a conjugate to indicate the level of testosterone present in the saliva sample. The concentration of testosterone is used to determine the likelihood of the saliva donors commitment to an interpersonal relationship.

### 1. RELATED APPLICATIONS

This application is a continuation in part of and claims benefit to U.S. non-provisional patent application Ser. No. 15/833,702 filed on 6 Dec. 2017 titled “Testosterone Saliva Test” that is incorporated herein in its entirety by reference and also related to provisional patent application 62/430,432 titled “Testosterone Saliva Test” filed on 6 Dec. 2016.

### BACKGROUND OF THE INVENTION

### 1. Field of the Invention

The present invention relates to human medical testing, and more particularly, to a non-invasive test to determine testosterone levels of men as part of a determination of their fidelity to interpersonal romantic relationships.

### 2. Description of the Related Art

Several designs for testosterone testing in humans have been designed in the past. None of them, however, includes a portable and simple lateral flow assay test that can nearly instantly provide empirical results of a man's estimated testosterone level.

Applicant believes that the closest reference corresponds to Hormones and Behavior, Burnham et al., issue 44 (2003) at page 119 journal article titled “Men in committed, romantic relationships have lower testosterone levels”. However, it differs from the present invention because the method of use and device in the present disclosure utilizes a lateral flow assay format with a control strip and a test strip that is used outside of laboratory controlled conditions and having a simplified and visually verifiable threshold test to determined estimated blood testosterone levels as they relate to evaluation of a man's commitment to a romantic relationship. Similarly, the present device may employ a single strip with both the control and test markers.

Other publications and patents describing the closest subject matter provide for a number of more or less complicated features that fail to solve the problem in an efficient and economical way. None of these disclosures suggest the novel features of the present invention.

### SUMMARY OF THE INVENTION

It is one of the main objects of the present invention to provide a simple and efficient device to test human testosterone levels derived from a saliva sample.

It is another object of this invention to provide a method of determining the level of commitment to a romantic relationship based at least in part estimated blood testosterone levels of a man while in a relationship.

It is still another object of the present invention to provide an accurate test to determine human testosterone levels using a calibrated test solution run in parallel to a saliva test. It should be appreciated that a single run could also include both the test and control features.

It is yet another object of this invention to provide such a device and method of use that is inexpensive to manufacture and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 shows a chart comparing salivary testosterone levels with relationship and familial status.

FIG. 2 shows a perspective view of an example of a testosterone saliva test.

FIG. 3 shows a perspective view of sample accessories to a testosterone saliva test.

FIG. 4 shows a plan view of an alternate version of a testosterone saliva test.

FIG. 5 shows a plan view of a testosterone saliva test.

FIG. 6 shows a plan view of a reader assembly internal components.

FIG. 7 shows an exploded perspective view of a cassette assembly.

### DETAILED DESCRIPTION OF THE PREFERRED EMBODIMENT

The subject device and method of use is sometimes referred to as the device, the invention, the test, the testosterone test, the saliva test, the machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Human male testosterone levels may reflect observable behavioral aspects relating to commitment to a romantic relationship. It has been shown that, among men generally, those involved in truly committed, romantic relationships have lower testosterone levels than men not involved in such relationships or who are contemplating divorce, are considering cheating or who are actually cheating. Studies show that men in committed, romantic relationships had on average about a twenty one percent lower testosterone level than men not involved in committed relationships. Testosterone levels of married men and unmarried men who were involved in committed, romantic relationships do not substantially differ, suggesting that male pair bonding status can be predicted by testosterone levels in that subject.

Differences in human male testosterone levels in the blood may be responsible for mating and parenting efforts expended by men toward their families. Both male-to-male competition and mate seeking appear in numerous studies are affected by testosterone hormone levels in the blood of men. By measuring the testosterone level, insight into the male behavior can be derived.

Referring now to the drawings, where the present invention is generally referred to with numeral 10, it can be observed that it basically includes a kit body 12, a well 14, a well 16, a pad 18, a pad 20, a strip 22, a strip 24, a conjugate 26 and a conjugate 28.

Generally, the kit body 12 is fabricated of a rigid and durable material. A plastic kit body 12 approximately 4 cm deep, 12 cm wide and 1 cm in height can be effective. These materials and dimensions are not intended to be limited, but merely a practical example of rough dimensions to aid the reader in understanding the scale of the device. Clearly, larger, smaller or differing materials of the kit body 12 will be effective and fall within the inventive scope of the device.

Formed into the kit body 12 is strip 22 that is substantially parallel to strip 24 and span the width of the device. A well 14 is placed on one end of the strip 24 and is dimensioned and adapted to accept a saliva sample. Similarly, a well 16 is located at one end of strip 24 and is dimensioned and adapted to receive a control sample specimen. The wells 14 and 16 are similarly dimensioned so they perform similarly. The strips 22 and 24 are also similarly dimensioned and positioned so they perform nearly the same as each other. An absorption pad, osmotic device, capillary action device or other saliva collection and delivery may be used alternately than the well 14 and strip 24.

When a sample of saliva is placed into well 14 it wicks along the strip 22. The saliva wicks through and past the conjugate 26 zone where the saliva reacts with a compound incorporated into the strip 22. The compound may be gold nanoparticles with antibodies that react in the presence of testosterone. Other chemistries of conjugate 26 can also be used depending on availability, sensitivity, cost and effectiveness as detecting trace amounts of testosterone in the saliva of the test subject.

At the other end of the strip 22 is a pad 18. The strip 24 also has a pad 20 opposite the well 16 end of the strip 24. The pads 18 and 20 absorb saliva and the control solution to ensure that the tested samples flow along the length of the respective strip 22 and strip 24. This ensures that reliably equal volumes of liquid tested will pass through the conjugates 26 and conjugate 28. The absorbent nature of the pads 18 and 20 help prevent overflow and spilling of tested fluids.

Similar to the saliva, a control test solution is placed into the well 16 as part of the test. The control solution wicks along the strip 24 through the conjugate 28 in the central section of the strip 24 as the control solution is drawn by wicking towards the pad 20 where any excess solution is absorbed. There may also be two wells that perform in parallel, each with a strip.

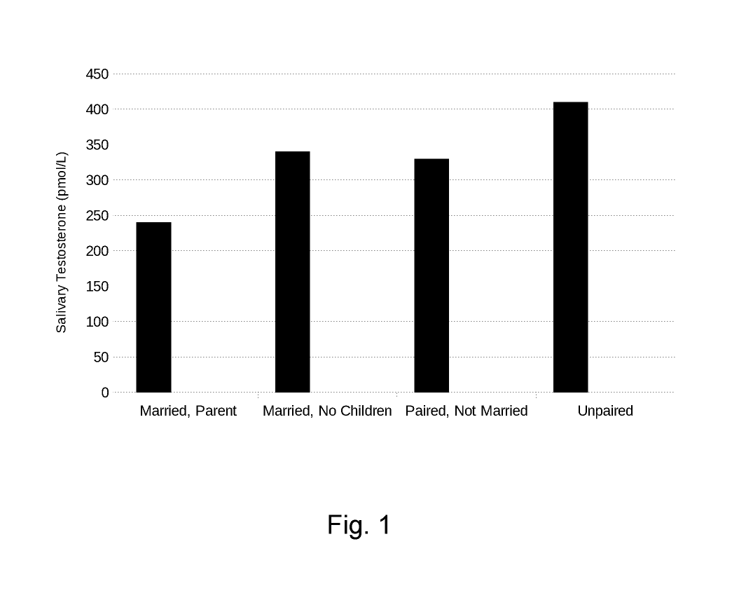

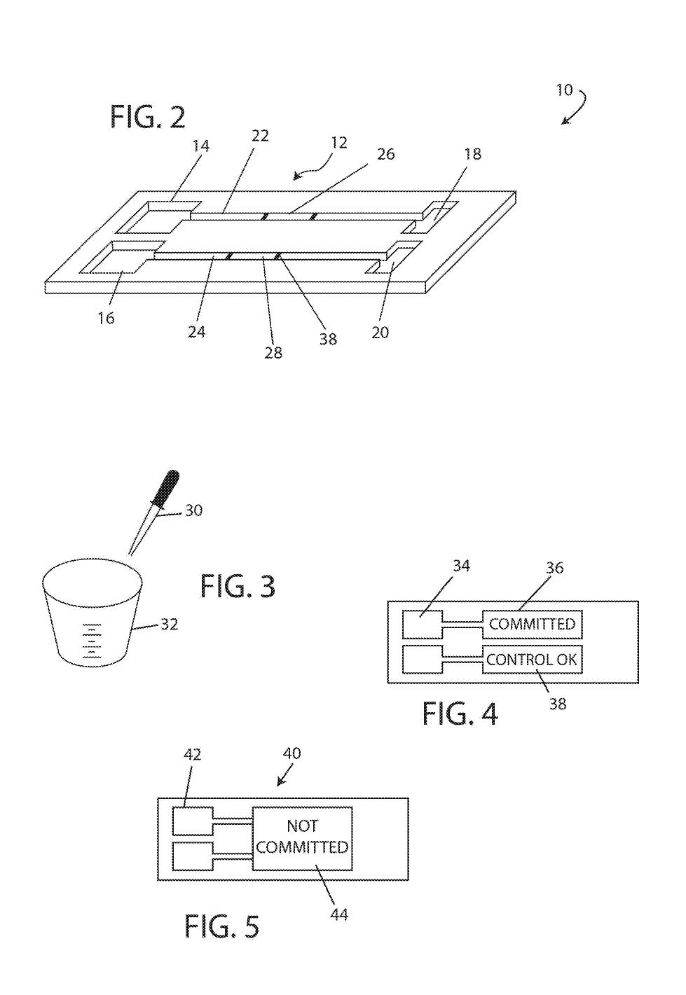

The control solution has a precisely calibrated concentration of testosterone or other appropriate analog of testosterone. For purposes outlined in the chart comprised of FIG. 1, the concentration of testosterone in the control solution should be equal to the testosterone found in saliva when a test subject would have about 360 pMol/L of testosterone in their blood. This test/device could be used for differing levels of concentrations that the user is desirous of measuring. FIG. 1 identifies several options.

To aid a user in reading the results of the test there may be one or more indicators similar to indicator 38. The indicator 38 may be a simple line or pair of lines that are revealed when a threshold concentration of testosterone is wicked through the conjugate 28 zone. If multiple indicators 38 are present it is possible to have different concentrations activate adjacent indicators 38. For example, a series of indicators 38 may be progressively along the strip 24 with one revealing itself at a first concentration (i.e. 300 pMol/L testosterone), the next indicating with a higher ratio (i.e. 360 pMol/L) and a third indicator turns darker or lighter only when presented with a higher ration of testosterone in the wicked fluid (i.e. 450 pMol/L). In this fashion a sample can be determined to be in a particular range. Of course the specific range points of the indicators 38 can be made to any desired concentration as would be appropriate for the specific test parameters. In at least one specific chemistry lightening of the indicator 38 progressively as higher concentrations of testosterone is found has been effective.

The control solution should be precisely calibrated to a specific concentration. For example, in the representative ratios above, the control solution could be precisely 360 pMol/L. An application of the control solution to the well 16 would wick the solution past the three indicators 38 and the first two would cause an indication but the 450 pMol/L indicator would not change because that concentration was not achieved in the test. Again, these are mere examples for a better understanding of a typical type of device and the specific numbers of indicators 38 and concentrations they alert at can be adjusted.

In another version, an indicator 38 may darken or indicate when it is moistened by the test fluid or saliva regardless of testosterone concentration and the second indicator 38 may be set to indicate a specific level of testosterone detected. In this example, the first indicator 38 the saliva or test fluid meets while wicking along the well 24 could be the one calibrated to alert at a specific testosterone concentration and the second indicator 38, nearer the pad 20, can alert on any moisture thereby proving that a sample of sufficient volume was put into the well to initiate the testing.

Generally, both sides of wells 14 and 16, pads 18 and 20, strips 22 and 24, conjugates 26 and 28 and any indicators 38 present will be the same for both sides of the kit body 12 so that either side can be equally used by the control solution or saliva interchangeably. In some cases the control solution (sometimes referred to as the test solution) will behave differently than the saliva so that each side of the test body 12 will not be identical but will have chemical differences to get the achieved results of reliably checking the control against the saliva sample to ensure repeatable and accurate results.

FIG. 3 shows accessories that may be included in a test kit to include, among other things and features, a pipette 30 and a cup 32. Depending on the saliva sample size, the size of the cup, the individual user or other factors, it may be necessary to expectorate saliva into a cup 32 instead of directly into the well 14.

A pipette 30 (or dropper, etc. . . . ) may also be provided with the cup to remove a precise volume of control solution or saliva into the wells 14 and 16. The use of a pipette 30 can also help avoid sampling of saliva with foam or other undesired parts. The pipette 30 can be precisely calibrated to pull a precise volume of fluid by capillary action to be dispensed into the wells 14 and 16 for the test.

FIGS. 4 and 5 show some alternative and optional features on variations of the inventive concept and include, among other features, a well 34, an indicator 36, an indicator 38, a kit body 40, a well 42 and an indicator 44. Any of these features and elements can be interchanged with other discussed elements to achieve the results of accurate saliva testing for testosterone.

In these variations, the well 34 or 42, pad, conjugate and strip, similar to those elements in the above described example are present. A difference is that instead of a line for an indicator 38, the indicator 36 can spell a word, like committed or the indicator 44 may read not committed. The indicator 38 for the control solution can emerge upon proper application of the control solution to prove that the device is functioning properly and that a saliva test in the other half of the device will be accurate when delivering results.

The present invention involves a test based on the detection of male salivary testosterone reaching a threshold level using lateral flow immunoassay.

Testosterone is a steroid hormone produced by men and women. Levels are generally naturally higher in men. Normal levels can range from about 260-1250 ng/dL. Studies found that a man's testosterone level dropped after marriage. A follow-up study at Harvard University found that a man's testosterone level didn't drop at the wedding ceremony. It dropped early with commitment. The study found that committed men had twenty one percent lower testosterone levels than men not involved in committed relationships. Further, it appears that the threshold for determining commitment is a testosterone level below 360 pMol/L. Another study from Harvard found that men in a committed relationship who were considering sexual interests outside of that relationship had elevated testosterone levels. Therefore, a test that could provide definitive, empirical evidence of male testosterone levels greater than about 360 pMol/L to determine whether that man is committed to the relationship, cheating or considering cheating or divorce.

Currently, this type of detection would require an enzyme-linked immunosorbent assay (ELISA) immunoassay. This can require several days and significant cost. For an ELISA test, a sample is typically sent to a lab using specialized instruments and highly trained personnel to operate the equipment.

Current rapid-result testosterone tests require a blood sample, which makes participation in the testing less appealing. Blood samples may also require a nurse or other trained technician to draw the blood sample and conduct the test. This is a less private situation than couples may want to undertake. A test is needed that can be performed without significant training and skill in the home of office of people taking and administering the test.

In order to overcome these drawbacks, the present test and method of testing provides a quantitative near-instant assay and detection of salivary testosterone utilizing lateral flow immunoassay. This can manifest, for example, in a competitive assay format or with a sandwich assay format. Monoclonal antibodies may be utilized that are similar to those used in an ELIZA test.

In one embodiment, the test strip is comprised of a membrane strip, a sample pad, a conjugate (detector) pad, an absorbent (sink) pad and an antibody and antigen reagents.

The sample may be provided by the test subject by spitting into a well on the kit body. The well's design will ensure a quantifiable amount of saliva will be deposited on the pad. In addition to the sample amount, another control fluid will be added and will run parallel to the sample amount. The control sample will contain a known amount of testosterone or testosterone analog to use as a comparison to the saliva sample from the test subject. A package may be provided that includes the test body, instructions and a vial of control sample for convenient testing wherever and whenever needed.

Continuing this example, both the well and the control will be in fluid communication with the membrane strip that is part of the lateral flow assay method and device. Basic components of lateral flow assay test bodies are commercially available and consist generally of a sample pad with a pretreatment buffer that may contain blockers to prevent errors from interference. The sample pad is separated from at nitrocellulose membrane by the conjugate pad. The conjugate may consist of latex beads, gold nanoparticles or colored reagent combined with antibodies or antigens. The amount of detector molecules is specifically calibrated in this test to measure the amount of testosterone desired. The antibody or antigen may be conjugated with a colorant such as latex beads, dyes or nanoparticles.

The test body configuration would lead the saliva sample to a membrane, for example a nitrocellulose membrane, that would wick into either a color reaction or a signal that a digital reader can read. This membrane is then compared to the results achieved by the control to verify the testosterone threshold for a positive result is met. If not, the test or reagents may be faulty and a new test should be considered to gain results within the predetermined margin of error for that test as determined during manufacturing.

The final piece would consist of an absorbent pad to prevent back flow of the liquid saliva or test (calibration) liquid from draining out of the test body. This can improve safety for contact with body fluids (saliva) and generally make a cleaner test procedure.

A related embodiment is to create a thermometer or tube-like device or absorbent pad that would wick the saliva directly from a man's mouth using capillary action. When the saliva reaches the top it interacts chemically with an indicator compound and would signal a positive or negative result, as appropriate. The test taker then would remove the apparatus, and place a cap over the tip. The cap would ensure applied pressure to release the sample from the capillary and release the control onto the sample pad.

In another version of the testosterone saliva test a single strip, well, pad and conjugate are used instead of requiring a pair having one for the saliva and the other for the control fluid. In this version only the saliva is used for the test. No verification of the accuracy of the test is required by use of a control fluid.

To control the flow of saliva and normalize the assay, the saliva receiving pad (or other analogous component described herein) may include a carrying fluid or pushing fluid that may combine with the saliva sample to effectively travel or flow in concert along the path of the assay over the test markers.

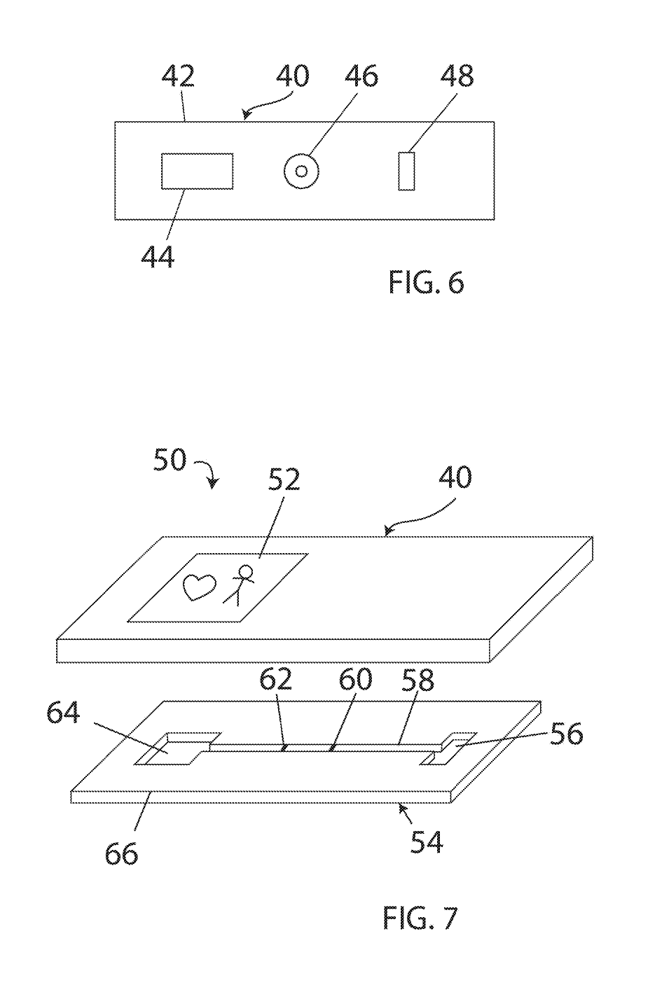

FIGS. 6 and 7 show a version of the test that also includes an electronic optical reader in combination with the lateral flow assay test to help with more precise and optionally quantitative results of testosterone concentration in the saliva sample. The cassette assembly 50 example includes, among other features, a reader assembly 40, a body 42, a processor 44, a camera 46, a battery 48, a display 52, an assay assembly 54, an absorption pad 56, a path 58, a test indicator 60, a control indicator 62, a saliva pad 64 and a tray 66.

The reader assembly 40 is a compact electronic device that is adapted to fit onto an assay assembly 54 combining to form the cassette assembly 50. The reader assembly 40 is powered by a battery 48 and controlled by a processor 44. The processor 44 and battery 48 work together with an integrated camera 46 on the interior surface. An electronic display 52 is on the exterior surface of the reader assembly 40.

The assay assembly 54 is similar to any of the versions disclosed herein. A saliva sample is placed onto the saliva pad 64 and travels along the path 58 towards the absorption pad 56. As the saliva moves over the control line 62 and test line 60 the reagents will react to predetermined degrees of dark or light depending on the concentration of testosterone in the saliva sample.

For some testosterone reagent combinations the absolute darkness of the test line 60 and control line 62 and relative darkness of the test line 60 and control line 62 are difficult to precisely determined with the human eye. To precisely and quantitatively determine the testosterone levels in the saliva the darkness of the test line 60 and control line 62 should be precisely measured.

To precisely measure the darkness of the test line 60 and control line 62 the reader assembly 40 is fit over the assay assembly 54 so that the camera 46 is positioned to optically view the test line 60 and control line 62. The tray 66 may be provided attached to the body 42 in one compact unit that protects the electronics of the reader assembly 40 and the reagents in the assay assembly 54.

The reader assembly 40 is turned on with a switch or is activated by the presence of the saliva sample on the saliva pad 64. The camera 46, controlled by the processor 44, reads the darkness of the test line 60 and control line 62. The darkness value of the control line 62 may be established as a datum for relative darkness of the test line 60. An empirical value for the darkness of the saliva reacted test line 60 may therefore be established. The darkness value may be a specific number value of darkness that may then be displayed on the display 52. In addition or alternatively, the display 52 may show an icon indicating whether the saliva sample donor is likely committed or not committed. In the example shown in FIG. 7 a heart icon indicates committed and a running man indicated not committed. Obviously, other appropriate icons could be equally effective act communication the determination of commitment. The processor 44 aids in analyzing the level of commitment based on pre-programmed software in the processor 44.

An important version of the invention can be fairly described as a human relationship commitment test. The test includes a lateral flow assay test style test integrated into a test body, for example a small plastic slab. The test body is adapted to accept a saliva sample that is drawn into the active areas of the test body. The test body has adhered to a test area a testosterone reactive conjugate. The saliva flows to the test area during the initial phases of the test. The test body has a control visual indicator that when reacted with the saliva sample proves visually that the testosterone reactive conjugate is reacting effectively with testosterone in the saliva sample. This could be a line appearing in the test area or other positive indicator. The test body has a test visual indicator that when reacted with the saliva sample visually indicates the concentration of testosterone in the saliva sample by varying intensity of the test visual indicator. This can be a lighter or darker line depending on the chemistry of the reagents. If the intensity of the test visual indicator is at or below a predetermined threshold then human relationship commitment is determined lacking because the testosterone exceeds a specific level. Likewise if the testosterone is determined to be below a threshold then commitment to the relationship is more likely established. Optionally, an electronic sensor is paired with the test body that optically determines an intensity of the test visual indicator relative to an intensity of the control visual indicator then calculates and displays in a human readable format a quantified concentration of testosterone in the saliva sample. This is essentially a camera that sees the changes to the test and control areas to measure the change in these areas after exposure to the saliva to determine the concentration of testosterone in the saliva with a high degree of quantitative accuracy.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

1. A human relationship commitment test comprised of:
a lateral flow assay test integrated into a test body;
the test body is adapted to accept a male only saliva sample;
the test body has adhered to a test area a testosterone reactive conjugate;
the test body has a control visual indicator that when reacted with the saliva sample proves visually that the testosterone reactive conjugate is reacting effectively with testosterone in the saliva sample;
the test body has a test visual indicator that when reacted with the saliva sample visually indicates the concentration of testosterone in the saliva sample by varying intensity of the test visual indicator;
if the intensity of the test visual indicator is at or below a predetermined threshold then human relationship commitment is visually determined to not be in a pursuit mode and if the intensity of the test visual indicator is above the predetermined threshold then the relationship commitment is determined lacking.

2. A human relationship commitment test as in claim 1 further characterized in that an electronic sensor is paired with the test body that optically determines an intensity of the test visual indicator relative to an intensity of the control visual indicator then calculates and displays in a human readable format a quantified concentration of testosterone in the saliva sample.
